(19) United States
(12) Patent Application Publication
(10) Pub. No.: US 2024/0058007 A1
(43) Pub. Date: Feb. 22, 2024

(54) CONVEYING APPARATUS CAPABLE OF REALIZING RAPID ELECTRICAL SEPARATION

(71) Applicant: Beijing Taijiewelye Technology Co., Ltd., Pinggu District, Beijing (CN)

(72) Inventors: Hang Tang, Pinggu District, Beijing (CN); Yongsong Xu, Pinggu District, Beijing (CN); Chuan Qin, Pinggu District, Beijing (CN); Jian Wu, Pinggu District, Beijing (CN); Lei Mu, Pinggu District, Beijing (CN); Xiuwen Wang, Pinggu District, Beijing (CN); Fei Chen, Pinggu District, Beijing (CN); Jinlei Wang, Pinggu District, Beijing (CN)

(21) Appl. No.: 18/259,952

(22) PCT Filed: Nov. 2, 2021
(86) PCT No.: PCT/CN2021/128038

§ 371 Date: Jun. 29, 2023

(30) Foreign Application Priority Data
Dec. 29, 2020 (CN) 202011585758.8

(51) Int. Cl. A61B 17/12

(52) U.S. Cl. CPC A61B 17/12022; A61B 2017/12063; A61B 17/00234

(57) ABSTRACT

A conveying apparatus includes a conveying catheter. An insulation catheter over the conveying catheter, a conductive catheter over the outside of the insulation catheter, and a support spring connected to the conveying catheter; the support spring includes a second cathode connected to the conveying steel catheter via a conductive wire; the second cathode includes a second anode provided with a separation point, and connected to the conductive catheter through an anode conductive wire; the anode conductive wire and the cathode conductive wire run through the conveying catheter; the second anode, the anode conductive wire and the conductive catheter form a positive path connected to a positive electrode; the second cathode, the cathode conductive wire and the conveying catheter form a negative path, connected to a negative electrode; and the second anode and the second cathode conduct the positive path and the negative path through electrolyte in blood.

### CROSS-REFERENCE TO RELATED APPLICATIONS

This application is a national phase entry under 35 U.S.C. § 371 of International Patent Application PCT/CN2021/128038, filed Nov. 2, 2021, designating the United States of America and published as International Patent Publication WO 2022/142706 A1 on Jul. 7, 2022, which claims the benefit under Article 8 of the Patent Cooperation Treaty of Chinese Patent Application Serial No. 202011585758.8, filed Dec. 29, 2020.

### TECHNICAL FIELD

The disclosure relates to the field of medical devices, and, in particular, to a conveying apparatus capable of realizing rapid electrical separation.

### BACKGROUND

Interventional embolization of aneurysms is a common method for treating aneurysms. Generally, a microcatheter is inserted into a root of the patient's thigh, and an embolic material is placed in an aneurysm cavity of an aneurysm along the microcatheter through a conveying apparatus, causing thrombus in the aneurysm cavity and reducing the blood flow in the aneurysm, thereby achieving the purpose of curing the aneurysm.

At present, after the embolic material is delivered into the aneurysm cavity through a conveying apparatus of the embolic material, the embolic material is generally separated from the conveying apparatus by means of electrical separation. The traditional electrical separation uses a human body as a circuit, an anode is connected to a power supply through a stainless steel conveying rod, a cathode is inserted into arms and other parts of the human body, and a connection between the cathode and the anode is realized through a long human blood vessel. The electrical separation is slow and takes about 30 s to 60 s. In a latest electrical separator, an anode is connected to a catheter by a conductive wire, however, a conveying rod and a support spring are used as a cathode to realize the conductive circuit. In this way, the separation time is reduced and takes about 10 s to 15 s, however, there is a problem of poor separation stability.

### BRIEF SUMMARY

The disclosure provides a conveying apparatus capable of realizing rapid electrical separation. In the conveying apparatus, a conveying steel catheter is used and an insulating catheter is sleeved on a proximal end of the conveying steel catheter; a conductive catheter is sleeved on the outer side of the insulating catheter; a second anode and a second cathode are both arranged at a distal end of the conveying steel catheter; the conductive catheter is connected to the second anode through an anode conductive wire wrapped with an insulating layer; an electrode end of the conveying steel catheter is connected to the second cathode through a cathode conductive wire wrapped with an insulating layer; a conductive path is realized through the second anode and the second cathode, which are very close to each other in distance therebetween, thereby greatly reducing the separation time; and by using the conductive wires wrapped with an insulating layer, the interference with charge flow can be reduced and separation stability can be improved.

To achieve the above object, there is provided a conveying apparatus capable of realizing rapid electrical separation, including:
a conveying steel catheter, wherein a proximal end of the conveying steel catheter is provided with a grinding part, an outer side of the grinding part is sleeved with an insulating catheter, the proximal end of the conveying steel catheter is connected to a first cathode of an external electrical separator;
a conductive catheter sleeved on an outer side of the insulating catheter, wherein the conductive catheter is connected to a first anode of the electrical separator;
a support spring connected to a distal end of the conveying steel catheter;
a second cathode located at a distal end of the support spring;
a cathode conductive wire inserted in the conveying steel catheter, wherein a proximal end of the cathode conductive wire is connected to the proximal end of the conveying steel catheter, a distal end of the cathode conductive wire is connected to the second cathode, and the second cathode, the cathode conductive wire and the conveying steel catheter form a negative path of the electrical separator;
a second anode located at a distal end of the second cathode, wherein the second anode is provided with a separation point; and
an anode conductive wire inserted in the conveying steel catheter, wherein a proximal end of the anode conductive wire is connected to the conductive catheter, a distal end of the anode conductive wire penetrates through the distal end of the support spring and is connected to the second anode, and the second anode, the anode conductive wire and the conductive catheter form a positive path of the electrical separator;
wherein the conveying steel catheter conveys the separation point to a location of a hemangioma, and an external implant connected to the separation point is conveyed into a hemangioma cavity, the electrical separator is turned on, the second anode and the second cathode connect the positive path and the negative path by means of electrolyte in blood to form a low-resistance circuit, and the separation point is electrochemically dissolved and fractured to be separated from an implant, and the implant remains within the hemangioma cavity.

Preferably, the conveying steel catheter is a taper-grinding stainless steel catheter or nickel-titanium catheter and has a diameter of 0.3 mm to 0.6 mm.

Preferably, the second anode is a stainless steel wire and has a diameter of 0.02 mm to 0.08 mm.

Preferably, the materials of the anode conductive wire and the cathode conductive wire are both noble metal alloy wires; and each of the noble metal alloy wires is externally provided with an insulating layer;
the diameters of the anode conductive wire and the cathode conductive wire are both within a range of 0.05 mm to 0.1 mm.

Preferably, the second cathode is ring-shaped or spring-shaped and has an outer diameter of 0.2 mm to 0.5 mm.

Further preferably, the material of the second cathode is a stainless steel or a platinum alloy.

Preferably, the support spring is made by winding a stainless steel wire with a diameter of 0.03 mm to 0.08 mm, and the support spring has an outer diameter of 0.2 mm to 0.6 mm.

Further preferably, the middle part of the support spring has a support core wire, and the support core wire is a taper-grinding stainless steel wire and has a diameter of 0.05 mm to 0.36 mm.

Preferably, a distance between the second anode and the second cathode is 0.1 mm.

An embodiment of the disclosure provides a conveying apparatus capable of realizing rapid electrical separation. In the conveying apparatus, a conveying steel catheter is used and an insulating catheter is sleeved on a proximal end of the conveying steel catheter; a conductive catheter is sleeved on the outer side of the insulating catheter; a second anode and a second cathode are both arranged at a distal end of the conveying steel catheter; the conductive catheter is connected to the second anode through an anode conductive wire wrapped with an insulating layer; an electrode end of the conveying steel catheter is connected to the second cathode through a cathode conductive wire wrapped with an insulating layer; a conductive path is realized through the second anode and the second cathode, which are very close to each other in distance therebetween, thereby greatly reducing the separation time; and by using the conductive wires wrapped with an insulating layer, the interference with charge flow can be reduced and separation stability can be improved.

### BRIEF DESCRIPTION OF THE DRAWINGS

FIG. 1 is a schematic structural diagram of a conveying apparatus capable of realizing rapid electrical separation according to an embodiment of the disclosure.

FIG. 2 is an enlarged view of part A in FIG. 1.

### DETAILED DESCRIPTION

The technical solutions of the disclosure will be further described in detail below through the accompanying drawings and embodiments.

An embodiment of the disclosure provides a conveying apparatus capable of realizing rapid electrical separation. In the conveying apparatus, a conveying steel catheter is used and an insulating catheter is sleeved on a proximal end of the conveying steel catheter; a conductive catheter is sleeved on the outer side of the insulating catheter; a second anode and a second cathode are both arranged at a distal end of the conveying steel catheter; the conductive catheter is connected to the second anode through an anode conductive wire wrapped with an insulating layer; an electrode end of the conveying steel catheter is connected to the second cathode through a cathode conductive wire wrapped with an insulating layer; a conductive path is realized through the second anode and the second cathode, which are very close to each other in distance therebetween, thereby greatly reducing the separation time; and by using the conductive wires wrapped with an insulating layer, the interference with charge flow can be reduced and separation stability can be improved.

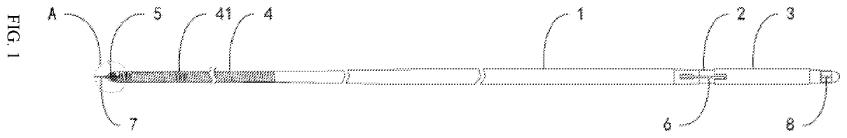

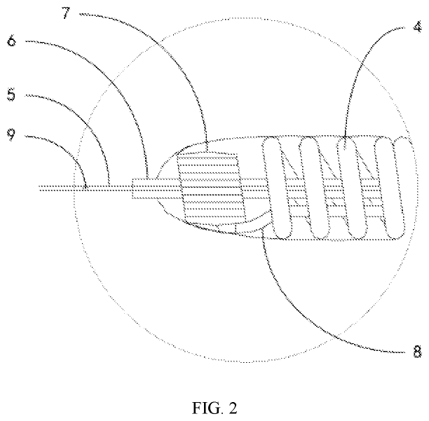

FIG. 1 is a schematic structural diagram of a conveying apparatus capable of realizing rapid electrical separation according to an embodiment of the disclosure, and FIG. 2 is an enlarged view of part A in FIG. 1. As shown in FIG. 1 and FIG. 2, the conveying apparatus capable of realizing rapid electrical separation includes: a conveying steel catheter 1, an insulating catheter 2, a conductive catheter 3, a support spring 4, a second anode 5, an anode conductive wire 6, a second cathode 7, a cathode conductive wire 8 and a separation point 9.

The conveying apparatus capable of realizing rapid electrical separation according to the embodiment of the disclosure can be used in an aneurysm surgery for conveying an embolic implant into an aneurysm cavity through a microcatheter.

The conveying steel catheter 1 is a taper-grinding stainless steel catheter or nickel-titanium catheter and has a diameter of 0.3 mm to 0.6 mm. In the embodiment, take hand-held end of an operator as a proximal end, the proximal end of the conveying steel catheter 1 has a grinding part, the insulating catheter 2 is sleeved on the grinding part, and the conductive catheter 3 is sleeved on an outer side of the insulating catheter 2, wherein the conductive catheter 3 is shorter than the insulating catheter 2.

A power supply used in the conveying apparatus capable of realizing rapid electrical separation according to the embodiment of the disclosure is an external electrical separator. A positive electrode of the electrical separator device is regarded as a first anode and a negative electrode of the electrical separator is regarded as a first cathode. When the proximal end of the conveying steel catheter 1 is inserted into a power supply port of the electrical separator, the conductive catheter 3 is connected to the first anode of the electrical separator, and the proximal end of the conveying steel catheter 1 is connected to the first cathode of the electrical separator.

The support spring 4 is connected to a distal end of the conveying steel catheter 1. The support spring 4 is a spring made by winding a stainless steel wire with a diameter of 0.03 mm to 0.08 mm. The outer diameter of the spring is 0.2 mm to 0.6 mm. The support spring 4 can soften the structure of the distal end of the conveying steel catheter 1, so that the conveying steel catheter 1 can fit the microcatheter better during the pushing process. A middle part of the support spring 4 also has a support core wire 41, which is a taper-grinding stainless steel wire. The support core wire 41 has a diameter of 0.05 mm to 0.36 mm. The support core wire 41 has a thin distal end, which is soft and smooth and a thick proximal end, which can improve the support stability.

The second cathode 7 is arranged at the distal end of the support spring 4, and the second anode 5 is arranged at the distal end of the second cathode 7. The second anode 5 is connected to the conductive catheter 3 by the anode conductive wire 6, and the conductive catheter 3, the anode conductive wire 6 and the second anode 5 form a positive path of the electrical separator. The second cathode 7 is connected to the proximal end of the conveying steel catheter 1 by the cathode conductive wire 8, and the conveying steel catheter 1, the cathode conductive wire 8 and the second cathode 7 form a negative path of the electrical separator. Both the anode conductive wire 6 and the cathode conductive wire 8 are inserted in the conveying steel catheter 1 and the support spring 4, wherein the anode conductive wire 6 penetrates through the distal end of the support spring 4 to be connected to the second anode 5.

The second anode 5 is a stainless steel wire with a diameter of 0.02 mm to 0.08 mm and has a separation point 9 thereon to be connected to the implant to be conveyed. The second cathode 7 is ring-shaped or spring-shaped and has outer diameter of 0.2 mm to 0.5 mm, and the material of second cathode 7 is a stainless steel wire or a platinum alloy. The distance between the second anode 5 and the second cathode 7 is 0.1 mm, so that the second anode 5 and the second cathode 7 can connect the positive path and the negative path of the electrical separator by means of electrolyte in the blood to form a low-resistance circuit. The separation point 9 has an electrochemical dissolution reaction and is electrochemically dissolved and fractured after is to 3s to be separated from the implant. In this way, the implant and the conveying apparatus can be separated rapidly and stably.

The anode conductive wire 6 and the cathode conductive wire 8 are both noble metal alloy wires having a diameter of 0.05 mm to 0.1 mm and externally provided with an outer insulating layer. The extremely small resistance of the noble metal alloy wire and the design of the external insulating layer cause less interference with the charge flow, thereby greatly improving the stability of electrical separation.

The embodiment of the disclosure provides the following working process.

An implant is connected to the conveying apparatus through the separation point 9, and after the conveying apparatus pushes the implant into a hemangioma cavity through a microcatheter, the proximal end of the conveying steel catheter 1 is inserted into the power supply port of the electrical separator, and the electrical separator is turned on, the second anode 5 and the second cathode 7 connect the positive path and the negative path by means of electrolyte in the blood to form a low-resistance circuit, and the separation point 9 is electrochemically dissolved and fractured to be separated from the implant, and the implant remains within the hemangioma cavity. In this way, the conveying and rapid separation of the implant is realized.

Relative to the prior art, the embodiment of the disclosure provides a conveying apparatus capable of realizing rapid electrical separation. In the conveying apparatus, a conveying steel catheter is used and an insulating catheter is sleeved on a proximal end of the conveying steel catheter; a conductive catheter is sleeved on the outer side of the insulating catheter; a second anode and a second cathode are both arranged at a distal end of the conveying steel catheter; the conductive catheter is connected to the second anode through an anode conductive wire wrapped with an insulating layer; an electrode end of the conveying steel catheter is connected to the second cathode through a cathode conductive wire wrapped with an insulating layer; a conductive path is realized through the second anode and the second cathode, which are very close to each other in distance therebetween, thereby greatly reducing the separation time; and by using the conductive wires wrapped with an insulating layer, the interference with charge flow can be reduced and separation stability can be improved.

The specific embodiments described above further explain the objects, technical solutions and beneficial effects of the disclosure. It should be understood that the above descriptions are only specific embodiments of the invention, and not intended to limit the scope of the disclosure. Any modifications, equivalents, improvements and the like made without departing from the spirit and principle of the disclosure should fall within the scope of the disclosure.

1. A conveying apparatus capable of realizing rapid electrical separation, comprising:
a conveying steel catheter, wherein a proximal end of the conveying steel catheter is provided with a grinding part, an outer side of the grinding part is sleeved with an insulating catheter, the proximal end of the conveying steel catheter is connected to a first cathode of an external electrical separator;
a conductive catheter sleeved on an outer side of the insulating catheter, wherein the conductive catheter is connected to a first anode of the electrical separator;
a support spring connected to a distal end of the conveying steel catheter;
a second cathode located at a distal end of the support spring;
a cathode conductive wire inserted in the conveying steel catheter, wherein a proximal end of the cathode conductive wire is connected to the proximal end of the conveying steel catheter, a distal end of the cathode conductive wire is connected to the second cathode, and the second cathode, the cathode conductive wire and the conveying steel catheter form a negative path of the electrical separator;
a second anode located at a distal end of the second cathode, wherein the second anode is provided with a separation point; and
an anode conductive wire inserted in the conveying steel catheter, wherein a proximal end of the anode conductive wire is connected to the conductive catheter, a distal end of the anode conductive wire penetrates through the distal end of the support spring and is connected to the second anode, and the second anode, the anode conductive wire and the conductive catheter form a positive path of the electrical separator;
wherein the conveying steel catheter conveys the separation point to a location of a hemangioma, and an external implant connected to the separation point is conveyed into a hemangioma cavity, the electrical separator is turned on, the second anode and the second cathode connect the positive path and the negative path by means of electrolyte in blood to form a low-resistance circuit, and the separation point is electrochemically dissolved and fractured to be separated from an implant, and the implant remains within the hemangioma cavity.

2. The conveying apparatus capable of realizing rapid electrical separation of claim 1, wherein the conveying steel catheter is a taper-grinding stainless steel catheter or nickel-titanium catheter and has a diameter of 0.3 mm to 0.6 mm.

3. The conveying apparatus capable of realizing rapid electrical separation of claim 1, wherein the second anode is a stainless steel wire and has a diameter of 0.02 mm to 0.08 mm.

4. The conveying apparatus capable of realizing rapid electrical separation of claim 1, wherein materials of the anode conductive wire and the cathode conductive wire are both noble metal alloy wires and each of the noble metal alloy wires is externally provided with an insulating layer; and
diameters of the anode conductive wire and the cathode conductive wire are both within a range of 0.05 mm to 0.1 mm.

5. The conveying apparatus capable of realizing rapid electrical separation of claim 1, wherein the second cathode is ring-shaped or spring-shaped and has an outer diameter of 0.2 mm to 0.5 mm.

6. The conveying apparatus capable of realizing rapid electrical separation of claim 5, wherein material of the second cathode is a stainless steel wire or a platinum alloy.

7. The conveying apparatus capable of realizing rapid electrical separation of claim 1, wherein the support spring is made by winding a stainless steel wire with a diameter of 0.03 mm to 0.08 mm, and the support spring has an outer diameter of 0.2 mm to 0.6 mm.

8. The conveying apparatus capable of realizing rapid electrical separation of claim 7, wherein a middle part of the support spring has a support core wire, and the support core wire is a taper-grinding stainless steel wire and has a diameter of 0.05 mm to 0.36 mm.

9. The conveying apparatus capable of realizing rapid electrical separation of claim 1, wherein a distance between the second anode and the second cathode is 0.1 mm.
